(19) United States
(12) Patent Application Publication
(10) Pub. No.: US 2006/0089507 A1
(43) Pub. Date: Apr. 27, 2006

(54) Method for separating isocyanates out from a reaction mixture

(75) Inventors: Martin Sohn, Mannheim (DE); Eckhard Stroefer, Mannheim (DE); Filip Nevejans, St. Gillis-Waas (DE); Ulrich Penzel, Tettau (DE); Hans-Jurgen Pallasch, Ludwigshafen (DE); Michael Sander, Limburgerhof (DE); Hans Volkmar Schwarz, Overijse (DE); Wolfgang Mackenroth, Bad Durkheim (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 10/538,119

(22) PCT Filed: Dec. 13, 2003
(86) PCT No.: PCT/EP03/14186

§ 371 Date: Jun. 7, 2005

(30) Foreign Application Priority Data
Dec. 19, 2002 (DE) 102 60 093.7

(51) Int. Cl. C07C 263/04

(52) U.S. Cl. 560/347

(57) ABSTRACT

The present invention relates to a process for preparing isocyanates by reaction of amines with phosgene in a reactor and subsequent separation of the isocyanate from the reaction mixture leaving the reactor and purification of the isocyanate, wherein the separation and purification of the isocyanate is carried out in a column having a pressure at the top of 1-950 mbar, preferably 5-50 mbar, particularly preferably 10-20 mbar, and a temperature at the bottom of 90-250° C., preferably 120-170° C., particularly preferably 130-150° C., and the pure isocyanate stream is preferably taken off in liquid or gaseous form at a side offtake of the column.

The present invention relates to a process for the separation of isocyanates from a reaction mixture and purification of the isocyanates in the preparation of aromatic or aliphatic isocyanates. In the case of aromatic isocyanates, these are preferably methylenedi(phenyl isocyanate) (MDI) and tolylene diisocyanate (TDI), while in the case of aliphatic isocyanates, preference is given to hexamethylene diisocyanate (HDI) and isophorone diisocyanate (IPDI).

The continuous preparation of organic isocyanates by reaction of primary organic amines with phosgene has been described many times and is carried out on a large industrial scale (cf., for example, Ullmanns Enzyklopädie der Technischen Chemie, Volume 7 (Polyurethane), 3^{rd} revised edition, Carl Hanser Verlag, Munich-Vienna, p. 76 ff (1993)). The aromatic isocyanates TDI (tolylene diisocyanate) and MDI (methylenedi(phenyl isocyanate)) or PMDI (polymethylenepolyphenylene polyisocyanate) and the aliphatic isocyanates HDI (hexamethylenedi(phenyl isocyanate)) and isophorone diisocyanate (IPDI), in particular, are produced on an industrial scale.

The preparation of isocyanates from the corresponding amines by phosgenation has hitherto mostly been carried out in stirred vessels as described, for example, in DE-A-1468445, in cascades of stirred vessels as described, for example, in DE-C 844896, in packed reaction columns or reaction columns as described, for example, in WO 99/5428 and DE-A-2112181 or in unpacked columns. Operation in the circulation mode is often necessary to achieve a sufficient residence time for complete conversion in a limited reaction volume (holdup). Since the reaction of amine and phosgene in the liquid phase is very fast, a mixing reactor in which the reaction stream passed through the mixing device is subjected to high shear is frequently used for the first reaction stage. Known mixing apparatuses include, in particular, nozzles such as annular slit nozzles, annular hole nozzles, smooth jet mixing nozzles, fan jet nozzles, angle jet chamber nozzles, three-fluid nozzles, countercurrent mixing chambers, holdup nozzles and Venturi mixing nozzles.

The first stage of the isocyanate synthesis is frequently carried out at very low temperature and the second stage is then carried out at significantly higher temperature in a residence apparatus. This process is frequently referred to as cold-hot phosgenation. A description may be found, for example, in W. Siefken, Liebigs Analen der Chemie 562 (1949), page 96. A suspension of the intermediates carbamoyl chloride and amine hydrochloride is firstly prepared at low temperature, usually at 0° C. or room temperature, at most 60° C., and this is then reacted at higher temperatures, usually from 100 to 200° C., in a residence apparatus to form the isocyanate. Such two-stage processes are described in Ullmanns Enzyklopädie der Technischen Chemie, Volume 7 (Polyurethane), 3^{rd} revised edition, Carl Hanser Verlag, Munich-Vienna, p. 76 ff (1993), and, for example, in the patent documents DE 2058032, DE 2153268 and DE 2908703.

As residence apparatuses, it is possible to use the reactors which are customary and known for the preparation of isocyanates and have been mentioned above by way of example.

The preparation of isocyanates is usually carried out in solution. As solvents for the preparation of isocyanates, preference is given to using chlorinated aromatic hydrocarbons such as dichlorobenzene, chlorobenzene, trichlorobenzene, or aromatic or aliphatic hydrocarbons such as toluene, xylene, benzene, pentane, hexane, heptane, octane, cyclohexane, biphenyl, ketones such as 2-butanone, methyl isobutyl ketone, esters such as diethyl isophthalates, ethyl acetate, butyl acetate, nitriles such as acetonitrile, or sulfolane, etc.

After the reaction is complete, the solvent, which generally has a boiling point lower than that of the isocyanate, is separated off from the isocyanate and any residue and is worked up by distillation. In the case of tolylene diisocyanate (TDI), the isocyanate is subsequently separated from the residue by distillation and is purified by distillation or by crystallization. (is also done, inter alia, by competitors in the case of TDI. We should not make it too obvious that we practice distillation). In addition, further separation operations can be carried out so as to separate the isomer mixture in the case of TDI or MDI or the oligomer mixture in the case of MDI into individual fractions having different isomer and oligomer compositions.

The mixture of phosgene and hydrogen chloride obtained in the reaction of aliphatic or aromatic amines with phosgene to give the corresponding isocyanates can contain more or less large amounts of solvent and is generally separated into hydrogen chloride, which is usually obtained in gaseous form, and a generally liquid mixiture of phosgene and any solvent. The phosgene or phosgene/solvent mixture is then recirculated to the reaction.

U.S. Pat. No. 3,410,888 describes a process for isolating an aromatic diisocyanate from a reaction mixture, in which the isocyanate has two phenyl rings and the isocyanate groups are bound to carbon atoms of different phenyl rings. This applies to 4,4′-, 2,4′- and 2,2′-methylenedi(phenyl isocyanate) (MDI) and mixtures of these isomers or polymethylenepolyphenylene polyisocyanate (PMDI). The process described there comprises firstly reacting an appropriate aromatic diamine with phosgene and separating off part of the aromatic isocyanate prepared in this way in the course of the removal of the solvent by distillation, secondly transferring the distillation residue (bottom product) to a second distillation apparatus which is configured as a vessel over whose interior surface the residue is distributed as a thin film and whose temperature and pressure are sufficient to effect vaporization of the isocyanate, and thirdly taking off the vapor, which is essentially rich in isocyanate, from this second distillation apparatus. The vapor is condensed and the isocyanate is stored. As possible distillation apparatuses, climbing film evaporators or falling film evaporators are mentioned by way of example. The solvent selected in the isocyanate synthesis usually has a boiling point lower than that of the isocyanate; it is preferably at least 30° C. lower. In the case of a smaller boiling point difference, part of the isocyanate prepared is separated off together with the solvent in the solvent removal. This is followed by distillation of the crude isocyanate obtained as residue in the thin film evaporator. Separating off part of the isocyanate in the solvent removal has the advantage that undesired intermediate boilers, possibly colored impurities or components whose boiling points are between that of the isocyanate and that of the solvent, are separated off together with the solvent in the solvent removal. The mixture of the part of the isocyanate which has been separated off and the solvent is then returned as feed stream to the solvent removal or is passed to a separate evaporation or fractional distillation to concentrate the isocyanate. The latter is then recycled as feed to the solvent removal.

A disadvantage of this process is the fact that part of the isocyanate is separated off in the solvent removal, which makes additional purification of the solvent by distillation necessary. If the solvent for the preparation of the amine solution contains isocyanate, ureas are formed on mixing amine and solvent and these, since they are solids, lead firstly to blockages and secondly to a poor product quality.

In the production of isocyanates on an industrial scale in world-scale plants, i.e. plants having a capacity of at least 160,000 metric tons per annum of isocyanate, large amounts of a high-boiling residue which is difficult to handle and expensive to dispose of are obtained. The residue comprises mostly by-products formed by oligomerization, polymerization or undesirable secondary and subsequent reactions. A significant reduction in the amount of high boilers obtained can therefore be achieved by minimizing the thermal stress and the residence time in the distillation columns, in particular those used for separating the isocyanate from the reaction mixture and purifying the isocyanate by distillation.

It is an object of the present invention to provide a process in which, particularly in large-scale plants, the separation of the isocyanate from the reaction mixture and the purification of the isocyanate by distillation are carried out under such conditions that the amount of heavy products obtained is minimized and the isocyanate is obtained in good quality.

It has been found that this object is achieved by a process for separating isocyanates from reaction mixtures and purifying the isocyanates by distillation in large-scale plants, in which reduced formation of high boilers can be achieved using less apparatus while maintaining the same purity of the desired isocyanate and which is described in more detail below.

The present invention accordingly provides a process for preparing isocyanates by reaction of amines with phosgene in a reactor, optionally separation of the solvent used and subsequent separation of the isocyanate from the reaction mixture and purification of the isocyanate, wherein the separation and purification of the isocyanate is carried out in a column having a pressure at the top of 1-950 mbar, preferably 5-50 mbar, particularly preferably 10-20 mbar, and a temperature at the bottom of 90-250° C., preferably 120-170° C., particularly preferably 130-150° C., and the column is operated with countercurrent flow of gas and liquid. The pure isocyanate stream is preferably taken off in liquid or gaseous form at a side offtake of the column.

A particularly small amount of heavy products is obtained when the residence time in the bottom of the column is not more than six hours, preferably not more than four hours, based on the product taken off at the bottom.

The reaction mixture is advantageously fed into the lower part of the column; the column can preferably also be equipped with only a pure enrichment section without a stripping section. Internals used are the known internals of distillation and rectification columns. It is possible to use, inter alia, a tray column or a packed column. Trays which can be used are, for example, sieve trays, valve trays, bubble cap trays or dual flow trays and types of packing which can be used are, for example, sheet metal packing, woven fabric packing or mesh packing of all types. The use of ordered packing is particularly advantageous since it produces a low pressure drop. Beds of random packing elements are less suitable, but are not ruled out in principle. Specific types of packing which can be used are, for example, Sulzer BX, Sulzer CY, Sulzer Mellapak, Sulzer Mellapak Plus, Montz A3, Glitsch 4A, Kühni Rombopak, and others. As circulation vaporizers at the bottom, it is in principle possible to use all types of vaporizer, with falling film evaporators, long tube evaporators or thin film evaporators being particularly advantageous since they enable vaporization to be achieved without stressing the product. It is advantageous for the column to be equipped with a vertical dividing wall. For energy reasons and to avoid stressing the product and thus minimize the formation of heavy products, it can be advantageous for a single-stage or multistage vaporization to be installed upstream of the column used according to the present invention. Intermediate vaporization is also advantageous. In the case of preliminary vaporization, the liquid feed is fed into a vaporizer and partly or entirely vaporized in this way. The vapor stream and any remaining liquid stream is/are fed to the column. In the case of intermediate vaporization, the liquid is taken appropriately from a tray or collector of the column and passed to a heat exchanger. Both preliminary vaporization and intermediate vaporization can have one or more stages. The condenser at the top can be external or can be integrated in the column. It is possible to use both shell-and-tube apparatuses and plate apparatuses.

In principle, the solvent used can still be present in the reaction mixture fed into the column used according to the present invention. However, it is advantageous for at least part of it to be separated off beforehand. This can be carried out, for example, in an upstream column or a similar separation apparatus.

At the top of the column, hydrogen chloride, phosgene, solvent, chlorinated by-products and inerts such as nitrogen and carbon dioxide, inter alia, are obtained.

The heavy product taken off at the bottom outlet of the column comprises high-boiling oligomeric and polymeric compounds, typically ureas, polyureas, isocyanurates, uretdiones, carbodiimides and also isocyanate which has not been separated off completely.

If the bottom product discharged from the column still contains isocyanate, this can advantageously be recovered from the residue by depleting it in a further apparatus, preferably a column, at a pressure of 1-500 mbar, preferably 5-25 mbar, and a temperature of 100-225° C., preferably 110-140° C., down to a concentration of <10% by weight based on the feed stream to the first column. The bottom output of this column can be worked up once more in order to recover further residual isocyanate from the heavy product. All isocyanate fractions obtained in this way can be fed back into the first column for purification of the isocyanate.

The process of the present invention is particularly useful for the work-up of tolylene diisocyanate (TDI), methylenedi(phenyl isocyanate) (MDI), hexamethylene diisocyanate (HDI) and isophorone diisocyanate (IPDI). Other isocyanates can in principle also be purified in this way.

This process is particularly well suited to the preparation of TDI. TDI in particular tends to form heavy products which are very difficult to handle and which may reduce the availability of TDI plants. The formation of solids can be suppressed considerably by operating the column for separating off the TDI according to the present invention. This effect is particularly noticeable in large-scale plants having a capacity of at least 160,000 metric tons per annum.

The invention is illustrated by the following example.

### EXAMPLE

A reaction product mixture from the synthesis of tolylene diisocyanate (TDI) from tolylenediamine (TDA) and phosgene, from which the solvent had been separated off, was fed into the lower part of a distillation column having a diameter of 50 mm. The column was packed with 12 sections of mesh packing (Kühni Rombopak 9M, length of a section=630 mm). The temperature at the bottom was 145° C. and the pressure at the top was 15 mbar abs. As vaporizer, use was made of a thin film evaporator. The composition of the feed (1.14 kg/h) was 1.1 kg/h (96.5% by weight) of TDI including high-boiling TDI homologues, 0.02 kg/h (1.8% by weight) of uretdione and 0.02 kg/h (1.8% by weight) of chlorinated by-products and small amounts of low boilers such as hydrogen chloride, phosgene and others. At a side offtake of the column, 1.0 kg/h (99.9% by weight) of TDI together with small amounts (0.001 kg/h, 0.1% by weight) of chlorinated by-products were taken off. At the top of the column, downstream of the top condenser, viz. a shell-and-tube apparatus having 13 tubes, 0.018 kg/h of low boilers, predominantly hydrogen chloride and phosgene, was taken off in gaseous form and passed to an alkaline scrub for disposal. The condensate of the vapors obtained in the heat exchanger was returned as runback to the top of the column. 0.12 kg/h of bottoms were taken off at the bottom of the column and passed to a single-stage evaporation carried out at 5 mbar and 115° C. 0.06 kg/h of TDI was taken off in vapor form, condensed and combined with the other TDI obtained at the side offtake of the first column. The tar-like residue which remained was passed to incineration.

1. A process for preparing tolylene diisocyanate by reaction of amines with phosgene in a reactor and subsequent separation of the isocyanate from the reaction mixture and purification of the isocyanate, wherein the separation and purification of the isocyanate is carried out in a column having a pressure at the top of 1-950 mbar, preferably 5-50 mbar, particularly preferably 10-20 mbar, and a temperature at the bottom of 90-250° C., preferably 120-170° C., particularly preferably 130-150° C., and the column is operated with countercurrent flow of gas and liquid, the pure isocyanate stream being taken off in liquid or gaseous form at a side offtake of the column and the residence time in the bottom of the column being not more than 6 hours, based on the product taken off at the bottom, and wherein the column has a vertical dividing wall.

2. The process according to claim 1, wherein the bottom product from the column still contains isocyanate which is depleted in a further apparatus at a pressure of 1-500 mbar, preferably 5-25 mbar, and a temperature of 100-225° C., preferably 110-140° C., down to a concentration of <10% by weight based on the feed stream to the first column.

3. The process according to claim 1, wherein the column is preceded by a single-stage or multistage vaporization.

4. The process according to claim 1, wherein an intermediate vaporization is carried out on the column.

5. The process according to claim 1, wherein a flow-through vaporizer, preferably a falling film evaporator, long tube evaporator or thin film evaporator, is used as vaporizer for the column, the preliminary vaporization and the intermediate vaporization.

6. The process according to claim 1, wherein the column is packed with sheet metal packing, woven fabric packing or mesh packing.

7. The process according to claim 1, wherein the residence time in the bottom of the column is not more than six hours, preferably not more than four hours, based on the product taken off at the bottom.

8. The process according to claim 1, wherein the reaction mixture is fed into the lower part of the first column for separating off the isocyanate.
